(19) United States
(12) Patent Application Publication
(10) Pub. No.: US 2024/0358332 A1
(43) Pub. Date: Oct. 31, 2024

(54) SYSTEM FOR DISPLAYING OXYGEN STATE INDICATIONS

(71) Applicant: Masimo Corporation, Irvine, CA (US)

(72) Inventors: Prashanth Rengaswamy Chandran, Irvine, CA (US); Anand Sampath, Irvine, CA (US); Keith Ward Indorf, Lake Elsinore, CA (US); Sebastian T. Frey, Laguna Niguel, CA (US); Bilal Muhsin, Irvine, CA (US)

(21) Appl. No.: 18/765,863

(22) Filed: Jul. 8, 2024

Related U.S. Application Data
(63) Continuation of application No. 17/218,746, filed on Mar. 31, 2021, now Pat. No. 12,059,274.
Application No. 17/218,746 is a continuation of application No. 16/175,474, filed on Oct. 30, 2018, now Pat. No. 10,987,066.
(60) Provisional application No. 62/579,575, filed on Oct. 31, 2017.

(51) Int. Cl. A61B 5/00; A61B 5/1455

(52) U.S. Cl. CPC A61B 5/7445; A61B 5/0082; A61B 5/14551; A61B 5/14552; A61B 5/6826; A61B 5/746; A61B 5/7435; A61M 2205/3306; A61M 2230/205

(57) ABSTRACT

A patient monitoring system can have a display screen or potion thereof with graphic user interface for displaying indications of a patient's oxygen state. The indications can include the patient's SpO₂, dissolved oxygen index, and/or an increasing or decreasing trend of dissolved oxygen index. The displays of oxygen state indications can be compact, and/or able to provide direct visual information to a user of various aspects of the patient's oxygen state. The display elements can be used to represent any other physiological parameters.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

The present application is a continuation of U.S. patent application Ser. No. 17/218,746, filed Mar. 31, 2021, entitled “SYSTEM FOR DISPLAYING OXYGEN STATE INDICATIONS,” which is a continuation of U.S. patent application Ser. No. 16/175,474, filed Oct. 30, 2018, now U.S. Pat. No. 10,987,066, issued Apr. 27, 2021, entitled “SYSTEM FOR DISPLAYING OXYGEN STATE INDICATIONS,” which claims priority benefit under 35 U.S.C. § 119 (c) to U.S. Provisional Application No. 62/579,575, filed Oct. 31, 2017, titled “SYSTEM FOR DISPLAYING OXYGEN STATE INDICATIONS,” incorporated herein by reference in its entirety.

### FIELD

The present invention relates to the field of noninvasive oxygen delivery measurement using optical based sensors, and in particular, to displaying indication(s) of a patient's oxygen state.

### BACKGROUND

The measurement of oxygen delivery to the body and the corresponding oxygen consumption by the body's organs and tissues is vitally important to medical practitioners in the diagnosis and treatment of various medical conditions. Oxygen delivery is useful, for example, during certain medical procedures, where artificially providing additional oxygen to the patient's blood stream may become necessary. Patients may need supplemental oxygen in surgery, conscious sedation, or the intensive care unit. For example, during an intubation procedure, the patient will stop breathing while the procedure is performed. The patient is typically provided with oxygen before the intubation procedure. However, because the patient stops breathing during an intubation procedure, the patient's blood oxygen saturation level can fall. The medical practitioner must ensure that the patient has sufficient reserves of oxygen in the system before intubation so that suffocation is avoided during the intubation procedure. At the same time, providing oxygen at a high pressure to a patient can have its own negative effects in some patients, some of which include damages to the patient's lungs, and in particular, to the alveoli structures in the lungs. In some patients, for example neonates, even oxygen levels on the high end of a normal oxygenation range can cause blindness.

### SUMMARY

When oxygen molecules come into contact with blood, the majority of the oxygen molecules are bound to the hemoglobin in red-blood cells and a small portion is dissolved directly in the blood plasma. The current standard of care is to measure oxygen delivery through the use of a pulse oximeter. Pulse oximeters noninvasively measure and display oxygen saturation (SpO₂), which is the percentage of the patient's hemoglobin bound to oxygen molecules.

Another possible indicator of oxygen delivery is the partial pressure of oxygen (PaO₂) in the arterial blood. However, there are currently no reliable ways to measure arterial PaO₂ noninvasively. Invasive PaO₂ measurements require a blood gas analysis. The analysis may be needed intermittently during a surgical procedure and can interrupt and/or delay the surgical procedure. The analysis may require expensive sensors. Invasive PaO₂ measurements may also carry serious side effects that can harm the health of a patient.

One of the challenges of commercially available physiological monitors that provide SpO₂ readings is that the relationship between a patient's PaO₂ and SpO₂ is not linear. Specifically, SpO₂ measured by pulse oximetry can be reported as high as about 98% even when PaO₂ is as low as about 70 mmHg. A patient can be in a hypoxic state when the patient's PaO₂ falls below about 80 mmHg. Therefore, SpO₂ may not provide advance warning of falling arterial oxygenation until the patient's PaO₂ is already in the hypoxia range, which may not be adequate for providing advance warning of impending hypoxia. In an effort to prevent hypoxia, clinicians can provide supplemental oxygen to maintain SpO₂ at greater than about 98% during surgery to provide a “safety cushion” of oxygenation in the event of unexpected changes in oxygen delivery. However, the “safety cushion” can result in significant hyperoxia. When the SpO₂ level reaches or is close to 100%, the PaO₂ level can continue to rise if oxygen continues to be dissolved in the plasma. SpO₂ is not able to inform clinicians or any other users of an increasing amount of oxygen dissolved in the patient's blood beyond 100% SpO₂, when substantially all the hemoglobin has been fully saturated with oxygen modules. Hyperoxia have its own negative effects, including those described above. Therefore, providing information of the patient's hypersaturation condition can be helpful in allowing the clinician to stop delivery of supplemental oxygen temporarily and resume oxygen delivery when it is safe to do so.

Pulse oximeters according to the present disclosure can provide an index indicative of oxygen dissolved in the blood (hereinafter referred to as the “dissolved oxygen index” or the “index”). Example methods of determining such an index are described in U.S. Pat. No. 9,131,881, titled “ ” HYPERSATURATION INDEX” and issued Sep. 15, 2015, the entirety of which is incorporated by reference herein. An example of the index is the Oxygen Reserve Index™ (ORi™). The index can add to or supplement information from SpO₂, and/or invasive PaO₂ measurements using other equipment. The index is configured to provide information about oxygen dissolved in a patient's blood, which can indicate the patient's oxygenation in a moderate hyperoxic range. The moderate hyperoxic range can be, for example, when the patient's PaO₂ is between about 100 mmHg to about 200 mmHg. The pulse oximeter can be a standalone device or docked to a multi-parameter medical hub. The index can be displayed on a display screen of the standalone device and/or on a display screen of the medical hub.

The present disclosure provides various displays of indications of a patient's oxygen state including a patient's blood oxygenation state, such as the index, a patient's impending hypoxia and/or hyperoxia, and/or SpO₂. For example, the displays can inform a user of the patient's index when SpO₂ is high, such as greater than about 98%. Displaying various oxygen state indications on the same screen can provide a more comprehensive picture of the patient's oxygen state than displaying only SpO₂. The displays can be useful for patients who are on supplemental oxygen therapy, a ventilator or closed-loop positive pressure delivery device, or for any other medical applications where a patient's oxygen state needs to be monitored.

Another one of the challenges of commercially available physiological monitors is the limited display screen size, when patient monitors continue to expand in the number and type of monitored parameters made available to a user for review. Although a user is able to conveniently access a large amount of a patient's data from a single display screen, the display can become cluttered with the large number of parameters. It can also be challenging for the user to quickly and accurately understand the various oxygen state parameters shown on the display screen, among other physiological parameters, so as to make decisions about how to care for the patient.

The displays of oxygen state indications according to the present disclosure can be compact, and/or able to provide direct visual information of various aspects of the patient's oxygen state to a user reading the display screen. Examples of the aspects of the patient's oxygen state can include graphs, symbols, numerical indications, and/or a combination thereof, of SpO₂, the index, such as ORi™, and/or an increasing or decreasing trend of the index.

A noninvasive patient monitoring system for providing an indication of a patient's oxygen state system for providing an indication of a patient's oxygen state can comprise one or more sensors for outputting signals in response to a plurality of a patient's physiological parameters; one or more signal processors configured to receive the signals and calculate a first indicator responsive to a percentage of hemoglobin molecules bound to oxygen, a second indicator responsive to a quantity of oxygen dissolved in the patient's blood and not bound to any hemoglobin molecule, the second indicator providing different information to a caregiver than the first indicator, and a third indicator responsive to an increasing or decreasing trend of the second indicator; and a display responsive to output of the one or more signal processors to display the first, second, and third indicators. The first indicator can comprise the patient's oxygen saturation. The first indicator can be displayed as a graph and/or a numerical value. The second indicator can comprise a noninvasive index of dissolved oxygen in blood of the patient. The second indicator can be displayed as a plurality of shapes, the number of shapes being displayed corresponding to the patient's noninvasive index of dissolved oxygen in the blood. The shape can be a rectangle, a circle, a triangle, or a diamond. No shape may be displayed when the first indicator is below about 98%. The third indicator can further comprise a rate of increase or decrease of the patient's noninvasive index of dissolved oxygen in the blood. The third indicator can be displayed as an arrow pointing at an angle, a direction of the arrow corresponding to the increasing or decreasing trend of the patient's noninvasive index of dissolved oxygen in the blood and a magnitude of the angle corresponding to the rate of increase or decrease of the patient's noninvasive index of dissolved oxygen in the blood. The arrow can be displayed as pointing generally horizontally when the first indicator is below about 98%. The second indicator can be displayed as a shape that is empty, partially filled, or fully filled, the amount of filling corresponding to the patient's noninvasive index of dissolved oxygen in the blood. The shape can be empty when the first indicator is below about 98%. The shape can comprise a circle, a triangle, a rectangle, or a diamond. The third indicator can be displayed as a pointer placed at an angle, the pointer coupled with the shape to form a dial, a clock direction of the pointer corresponding to the increasing or decreasing trend of the patient's noninvasive index of dissolved oxygen in the blood and/or the rate of increase or decrease of the patient's noninvasive index of dissolved oxygen in the blood. The dial can display about 3 o'clock when the first indicator is below about 98%. The one or more sensors can comprise an optical sensor having at least one light emitter emitting light of a plurality of wavelengths into the body of the patient and a light detector detecting the light after attenuation of the body, wherein the attenuation can be responsive to oxygenation of the patient's blood. The first indicator can be determined based at least in part on the detected light. The second indicator can be determined based at least in part on the detected light. The third indicator can be determined based at least in part on the detected light.

A noninvasive patient monitoring system for providing an indication of a patient's oxygen state system for providing an indication of a patient's oxygen state can comprise at least one light emitter emitting light of a plurality of wavelengths into the body of the patient; a light detector detecting the light after attenuation of the body, wherein the attenuation can be responsive to oxygenation of the patient's blood, and outputting one or more signals from the light detector, the one or more signals responsive to said attenuation; one or more signal processors configured to process the one or more signals to electronically calculate a first indicator responsive to a quantity of oxygen dissolved in the patient's blood and not bound to any hemoglobin molecule, and a second indicator responsive to an increasing or decreasing trend of the first indicator; and a display responsive to output of the one or more signal processors to display the first and second indicators. The one or more signal processors can be further configured to calculate a third indicator responsive to a percentage of hemoglobin molecules bound to oxygen, the third indicator providing different information to a caregiver than the first or second indicator, and the display can be configured to display the third indicator. The third indicator can comprise the patient's oxygen saturation. The third indicator can be displayed as a graph and/or a numerical value. The first indicator can comprise the patient's noninvasive index of dissolved oxygen in the blood. The first indicator can be displayed as a plurality of shapes, the number of shapes being displayed corresponding to the patient's noninvasive index of dissolved oxygen in the blood. The shape can be a rectangle, a circle, a triangle, or a diamond. No shape may be displayed when the patient's oxygen saturation is below about 98%. The second indicator can further comprise a rate of increase or decrease of the patient's noninvasive index of dissolved oxygen in the blood. The second indicator can be displayed as an arrow at an angle, a direction of the arrow corresponding to the increasing or decreasing trend of the patient's noninvasive index of dissolved oxygen in the blood and a magnitude of the angle corresponding to the rate of increase or decrease of the patient's noninvasive index of dissolved oxygen in the blood. The arrow can be displayed as pointing generally horizontally when the first indicator is below about 98%. The first indicator can be displayed as a shape that is empty, partially filled, or fully filled, the amount of filling corresponding to the patient's noninvasive index of dissolved oxygen in the blood. The shape can be empty when the patient's oxygen saturation is below about 98%. The shape can comprise a circle, a triangle, a rectangle, or a diamond. The second indicator can be displayed as a pointer directed at an angle, the pointer coupled with the shape to form a dial, a clock direction of the pointer corresponding to an increasing or decreasing trend of the patient's noninvasive index of dissolved oxygen in the blood and/or the rate of increase or decrease of the patient's noninvasive index of dissolved oxygen in the blood. The dial can display about three o'clock when the first indicator is below about 98%.

A noninvasive patient monitoring system for providing an indication of a physiological parameter can comprise one or more sensors for outputting signals in response to a patient's physiological conditions; one or more signal processors configured to receive the signals and calculate a first indicator responsive to a magnitude of a physiological parameter, and a second indicator responsive to an increase or decrease of the physiological parameter; and a display responsive to output of the one or more signal processors to display the first and second indications. The second indicator can be further responsive to a rate of change of the physiological parameter. The first indicator can comprise a plurality of shapes, the number of shapes displayed corresponding to the magnitude of the physiological parameter. No shape may be displayed when the physiological parameter is zero or cannot be calculated. The shape can comprise a rectangle, a circle, a triangle, or a diamond. The second indicator can comprises an arrow. An angle of the arrow can correspond to the rate of change of the physiological parameter. The first indicator can comprise a shape that is empty, partially filled, or fully filled, the amount of filling corresponding to the magnitude of the physiological parameter. The shape can be empty when the physiological parameter is zero or cannot be calculated. The shape can comprise a circle, a triangle, a rectangle, or a diamond. The second indicator can comprise a pointer, the pointer coupled with the shape to form a dial, a clock direction of the pointer corresponding to the increase or decrease of the physiological parameter and/or the rate of change of the physiological parameter. The physiological parameter can be a patient's noninvasive index of dissolved oxygen in blood.

A noninvasive patient monitoring system for providing an indication of a patient's oxygen state system for providing an indication of a patient's oxygen state can comprise at least one light emitter emitting light of a plurality of wavelengths into the body of the patient, a light detector detecting the light after attenuation of the body, wherein the attenuation is responsive to oxygenation of the patient's blood, and outputting one or more signals from the light detector, the one or more signals responsive to said attenuation, and one or more signal processors configured to process the one or more signals to electronically calculate an indicator responsive to a quantity of oxygen dissolved in the patient's blood and not bound to any hemoglobin molecule and output an alert of the first indicator plateauing at a maximum value in response to an increase in oxygen supply to the patient. The system can further comprise a display screen responsive to output of the one or more signal processors to display the first indicator. The display can be further configured to display the alert. The system can also be configured to output the alert in an audio form. The first indicator can comprise a plurality of shapes, the number of shapes displayed corresponding to the magnitude of the quantity of oxygen dissolved in the patient's blood and not bound to any hemoglobin molecule. The first indicator can further comprise an indication of an increasing or decreasing trend of the magnitude. The increase in oxygen supply to the patient can be determined by monitoring the patient's FiO₂.

### BRIEF DESCRIPTION OF THE DRAWINGS

The drawings and following associated descriptions are provided to illustrate embodiments of the present disclosure and do not limit the scope of the claims. Corresponding numerals indicate corresponding parts, and the leading digit of each numbered item indicates the first figure in which an item is found.

FIG. 1A illustrates a perspective view of a patient monitoring system having a user-interface displaying a patient's physiological parameter(s).

FIG. 1B illustrates a block drawing of an example patient monitoring system.

FIG. 2A illustrates a perspective view of an example medical monitoring hub having a user-interface displaying a patient's physiological parameter(s).

FIG. 2B illustrates an example display screen of a medical monitoring hub.

FIG. 3 illustrates an example oxygen dissociation curve of a human patient.

FIG. 4 illustrates an example graph of SpO₂ and a patient's hypersaturation indication versus the R/IR ratio.

FIGS. 5A-5F illustrate example display screens or potion thereof with graphic user interface for displaying indications of various oxygen states.

FIGS. 6A-6F illustrate additional example display screens or potion thereof with graphic user interface for displaying indications of various oxygen states.

FIG. 7A illustrates an example flow chart for determining a “RESERVE FULL” status of the oxygen states.

FIGS. 7B and 7C illustrate example display screens or potion thereof with graphic user interface for displaying the “RESERVE FULL” status of the oxygen states.

### DETAILED DESCRIPTION

Aspects of the disclosure will now be set forth in detail with respect to the figures and various embodiments. One of skill in the art will appreciate, however, that other embodiments and configurations of the devices and methods disclosed herein will still fall within the scope of this disclosure even if not described in the same detail as some other embodiments. Aspects of various embodiments discussed do not limit the scope of the disclosure herein, which is instead defined by the claims following this description.

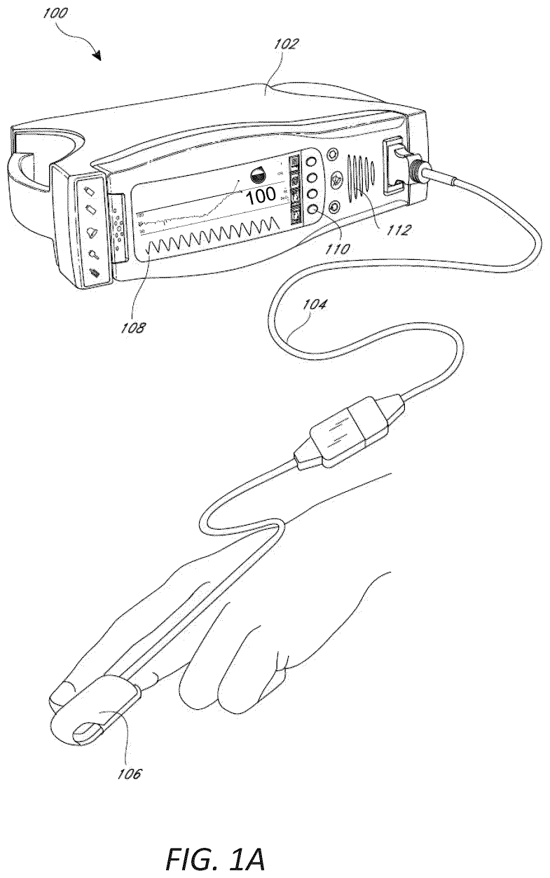

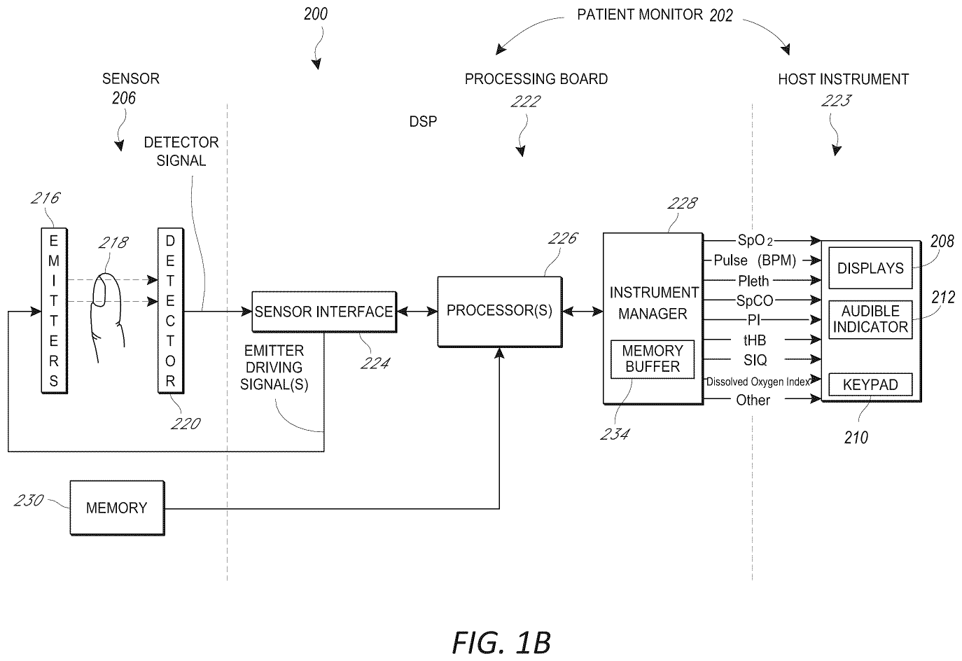

FIG. 1A illustrates an example patient monitoring system 100. The patient monitoring system 100 can include a patient monitor 102 attached to a sensor 106 by a cable 104. The sensor 106 can be an optical based sensor including one or more optical emitters and one or more optical detectors. In addition to the sensor 106 as illustrated in FIG. 1A, other types of sensor(s) that can be coupled to the patient monitor 102. The sensor 106 can monitor various physiological data of a patient and send signals indicative of the parameters to the patient monitor 102 for processing. For example, the one or more optical detectors can detect a plurality of wavelengths from the emitters after attenuation by the patient's body tissue. The patient monitoring system 100 can monitor SpO₂, indications of hypersaturation, perfusion index (PI), pulse rate (PR), hemoglobin count, and/or other parameters based at least in part on the signals received from the sensor(s).

The patient monitor 102 can include a display 108, control button(s) 110, and a speaker 112 for audible alerts. The display 108 can include a graphic user-interface, which can be capable of displaying readings of various monitored patient parameters. The readings can include numerical readouts, graphical readouts, animations, and/or the like. The display 108 can include a liquid crystal display (LCD), a cathode ray tube (CRT), a plasma screen, a Light Emitting Diode (LED) screen, Organic Light Emitting Diode (OLED) screen, or any other suitable display.

FIG. 1B illustrates details of a patient monitoring system 200 in a schematic form. A sensor 206 can include energy emitters 216 and one or more detectors 220. The energy emitters can be located on one side of a patient monitoring site 218. The one or more detectors 220 can be located generally opposite the energy emitters 216. The patient monitoring site 218 can be a patient's finger as shown in FIG. 1B. The patient monitoring site 218 can also be the patient's toe, car lobe, and/or the like. The energy emitters 216 can include LEDs or any other suitable optical emitters. The energy emitters 216 can emit various wavelengths of electromagnetic energy through the flesh of a patient at the monitoring site 218. The emitted wavelengths can be in the red and infrared ranges. The electromagnetic energy signals can be attenuated as the signals travel through the flesh of the patient at the monitoring site 218. The detector(s) 220 can detect the attenuated energy and send representative signals in response to the detected energy to the patient monitor 202 for processing. The patient monitor 202 can include a processing board 222 and a host instrument 223. The processing board 222 can include a sensor interface 224, signal processor(s) 226, and an instrument manager 228.

The host instrument can include one or more displays 208, a keypad 210, a speaker 212 for audio messages, and a wireless signal broadcaster 234. The keypad 210 can comprise a full keyboard, a track wheel, control buttons, and the like. The patient monitor 202 can also include buttons, switches, toggles, check boxes, and the like implemented in software and actuated by a mouse, trackball, touch screen, joystick, or other input device.

The sensor interface 224 can receive the signals from the sensor detector(s) 220 and pass the signals to the processor(s) 226 for processing into representations of physiological parameters. The representations of physiological parameters can be passed to the instrument manager 228. The instrument manager 228 can further process the parameters for display by the host instrument 223. The processor(s) 226 can also communicate with a memory 230 located on the sensor 106. The memory 230 can contain information related to properties of the sensor 106 that can be used during processing of the signals. A non-limiting example of information stored in the memory 230 can be the wavelengths emitted by the emitter 216. The various elements of the processing board 222 described above can provide processing of the detected signals. Tracking medical signals can be difficult because the signals may include various anomalies that do not reflect an actual changing patient parameter. The processing board 222 can apply filters and/or algorithms to detect truly changing conditions from limited duration anomalies. The host instrument 223 can display one or more physiological parameters according to instructions from the instrument manager 228. Additionally, the physiological parameter measurements can be sent to a remote server (such as a cloud server) or to a remote monitoring device, such as a cell phone, tablet, or laptop. The patient monitoring system can include a data transmitter, such as a wireless transmitter or transceiver, for electrically communicating with the remote server or remote monitoring device.

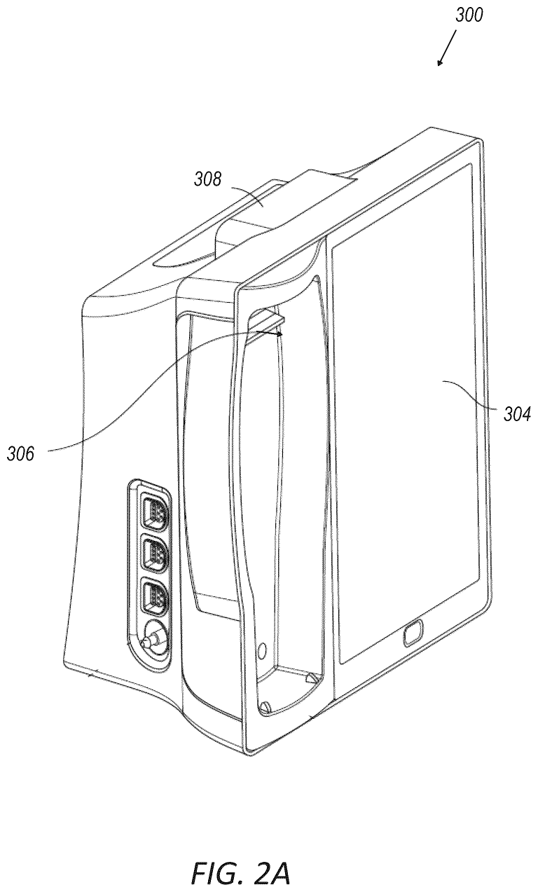

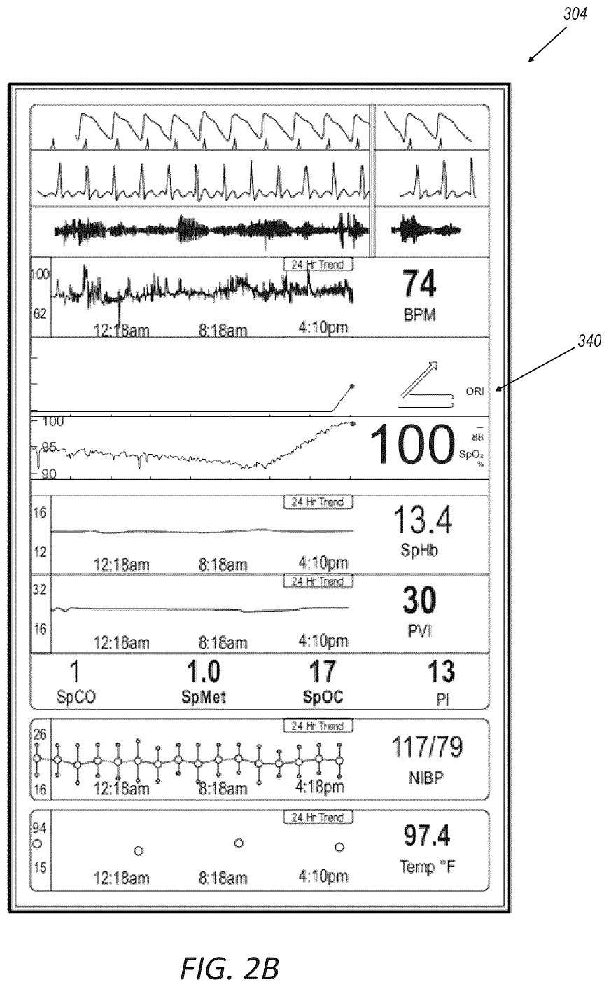

FIG. 2A illustrates a perspective view of an exemplary medical monitoring hub 300. The hub 300 can include a display 304 and a docking station 306. The docking station can be configured to mechanically and electrically mate with a portable patient monitor, such as the patient monitor 102, 202 in FIGS. 1A-1B. The display 304 can present various measurement and/or treatment data in numerical, graphical, waveform, or other display indicia 310, such as illustrated in FIG. 2B.

The display 304 can occupy a portion of a front face of the housing 308, or comprise a tablet or tabletop horizontal configuration, a laptop-like configuration or the like. Display information and data can optionally be communicated to a table computer, smartphone, television, or any other display system. The hub 300 can receive data from a patient monitor while docked or undocked from the hub.

Examples of patient monitors that can be docked to the hub 300 can include oximeters or co-oximeters, which are can provide measurement data for a large number of physiological parameters derived from signals output from optical and/or acoustic sensors, electrodes, and/or the like. The physiological parameters can include oxygen saturation, a dissolved oxygen index (such as, for example, ORi™), carboxy hemoglobin, methemoglobin, total hemoglobin, glucose, pH, bilirubin, fractional saturation, pulse rate, respiration rate, components of a respiration cycle, indications of perfusion including perfusion index, signal quality and/or confidences, plethysmograph data, indications of wellness or wellness indexes or other combinations of measurement data, audio information responsive to respiration, ailment identification or diagnosis, blood pressure, patient and/or measurement site temperature, depth of sedation, organ or brain oxygenation, hydration, measurements responsive to metabolism, or combinations of thereof. The hub 300 can also optionally output data sufficient to accomplish closed-loop drug administration in combination with infusion pumps or the like. Additional details of the medical hub 300 are described in U.S. application Ser. No. 14/512,237, filed Oct. 10, 2014 and titled “SYSTEM FOR DISPLAYING MEDICAL MONITORING DATA,” the entirety of which is incorporated herein by reference and should be considered a part of the specification.

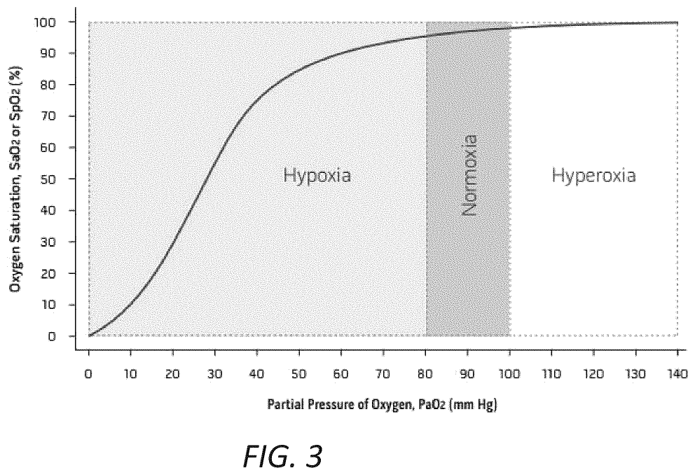

FIG. 3 illustrates the nonlinear relationship between the oxygen saturation and the partial pressure of oxygen in blood. The graph in FIG. 3 illustrates how oxygen saturation in the blood, SpO₂, or in the arterial blood, SaO₂, changes with respect to the PaO₂ in the blood. In this disclosure, SpO₂ is used interchangeably with SaO₂. A patient can be in a hypoxic state (less than normal oxygenation) when the patient's PaO₂ is below about 80 mmHg. A patient can be in a normoxic state (normal oxygenation) when the patient's PaO₂ is between about 80 mmHg and about 100 mmHg. A patient can be in a hyperoxic state (higher than normal oxygenation) when the patient's PaO₂ exceeds about 100 mmHg. A patient can be in a moderate hyperoxic state when the patient's PaO₂ is between about 100 mmHg to about 200 mmHg.

As shown in FIG. 3, SpO₂ initially increases as PaO₂ in the blood increases. After the SpO₂ and/or SaO₂ level(s) reach about 100%, the PaO₂ level continues to rise, but the SpO₂ and/or SaO₂ level off. As a result, SpO₂ measured using pulse oximetry can allow a user to monitor a patient's arterial blood oxygenation in hypoxia and/or normoxia, but not in hyperoxia. In addition, the increase in SpO₂ is small when a patient is in the normoxic and hyperoxic states. The SpO₂ reading can exceed about 95%, and be as high as about 98%, when PaO₂ is as low as about 70 mmHg. Therefore, when PaO₂ is transiting from the normoxic range to the hyperoxic range, SpO₂ reading may not be sensitive enough to provide advance warning of impending hypoxia. The patient can quickly be in hypoxia after the patient's dissolved oxygen is depleted without warning to the user. It can be too late when the user realizes that the patient is in hypoxia. The patient may continue to have insufficient oxygen until an increased delivery of oxygen brings the patient's oxygenation back to the normoxic range.

As described above, clinicians can deliver oxygen to maintain the patient's SpO₂ at greater than about 98% during surgery to have some reserve of oxygen in the blood in the event of unexpected changes in oxygen delivery. Such changes can occur due to, for example, cardiac depression, rapid hemorrhage, and/or interrupted ventilation. However, too much excess oxygen above about 98% SpO₂ can result in significant hyperoxia, which cannot be known from SpO₂ readings.

The patient monitoring system can be configured to measure the patient's hypersaturation conditions, that is, when the patient is in hyperoxia, in addition to monitoring SpO₂ and/or invasive PaO₂ measurements. The patient monitoring system can also be configured to inform a user of the patient's impending hypoxia. The patient monitoring system can be the patient monitor 102, 202 of FIGS. 1A and 1B, the patient monitor configured to be used with the medical hub 300 of FIGS. 2A and 2B, or any other patient monitoring devices.

As an example, the hypersaturation conditions can be monitored using pulse oximetry according to the present disclosure. Specifically, pulse oximetry can be used to determine a dissolved oxygen index in addition to SpO₂. The index is a dimensionless and continuous parameter configured to provide information about a patient's reserve of oxygen dissolved in the blood stream. This index can provide an indication of the patient's oxygenation in the moderate hyperoxic range. The index can assist a user, such as a medical practitioner in exercising her judgment in ensuring that the patient's blood is not overly hypersaturated with oxygen.

The index can be determined because the balance between oxygen supply and demand can alter venous oxygen saturation. As oxygen supply rises, venous oxygen saturation also increases if the patient's oxygen consumption is stable. During situations in which the SpO₂ level is at substantially 100%, the patient's oxygen consumption is stable if hemoglobin count and cardiac output are stable. The patient's oxygen consumption is substantially stable during anesthesia, surgery and some other procedures where the patient's oxygen state needs to be monitored. Change in venous oxygen saturation can result in changes in background light absorption at the plurality of wavelengths emitted by an optical sensor of a pulse oximeter in the presence of hyperoxia. Examples of the plurality of emitted wavelengths can include red and infrared wavelengths. The ratios of light absorption at the plurality of emitted wavelengths can be mapped at varying degrees of hyperoxia to allow calculation of the index based on SvO₂, which is the patient's oxygen saturation in the venous blood. Changes in SvO₂ can be observed when the patient is in moderate hyperoxia as defined herein and can be used to determine the index.

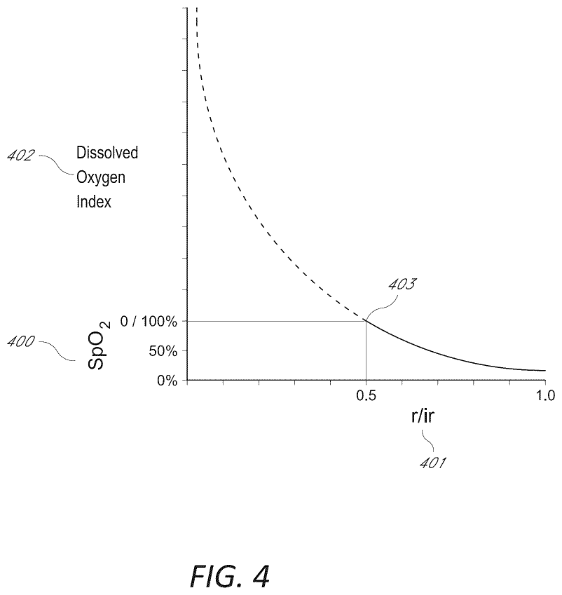

FIG. 4 illustrates an example graph of SpO₂ 400 and the index 402 versus the red to infrared (R/IR) ratio 401. In the illustrated example, the R/IR ratio 401 is at about 0.5 when the SpO₂ level 400 is at about 100%. Although the SpO₂ level 400 maxes out at 100% saturation, the R/IR ratio 401 continues to decrease when more oxygen is dissolved in the blood. The index 402 can be determined based on the R/IR ratio after the point 403, where the R/IR ratio translates to a SpO₂ level of 100% saturation. The index can also be determined when the SpO₂ level is slightly below 100% saturation. For example, the index can be determined when the SpO₂ level exceeds about 95%, about 97%, or about 98%.

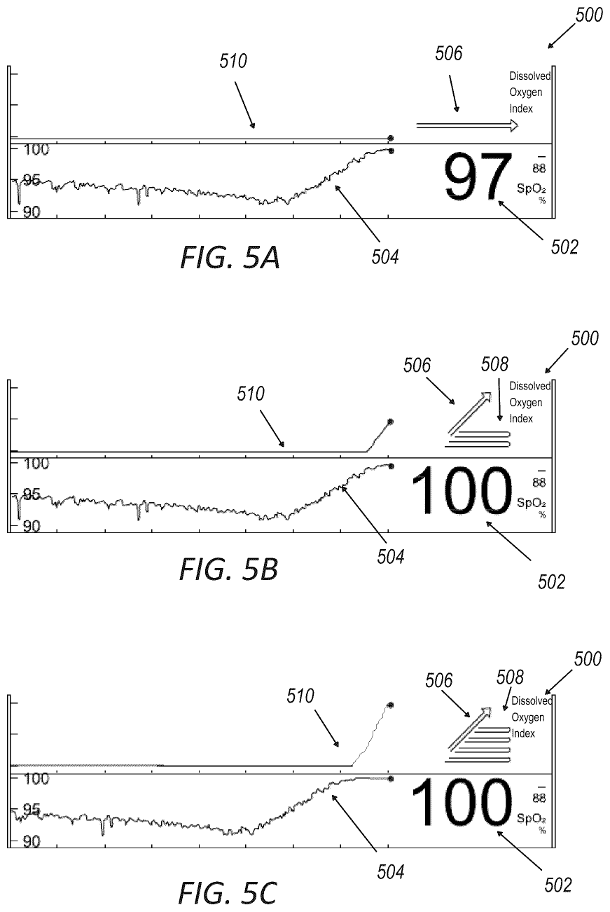

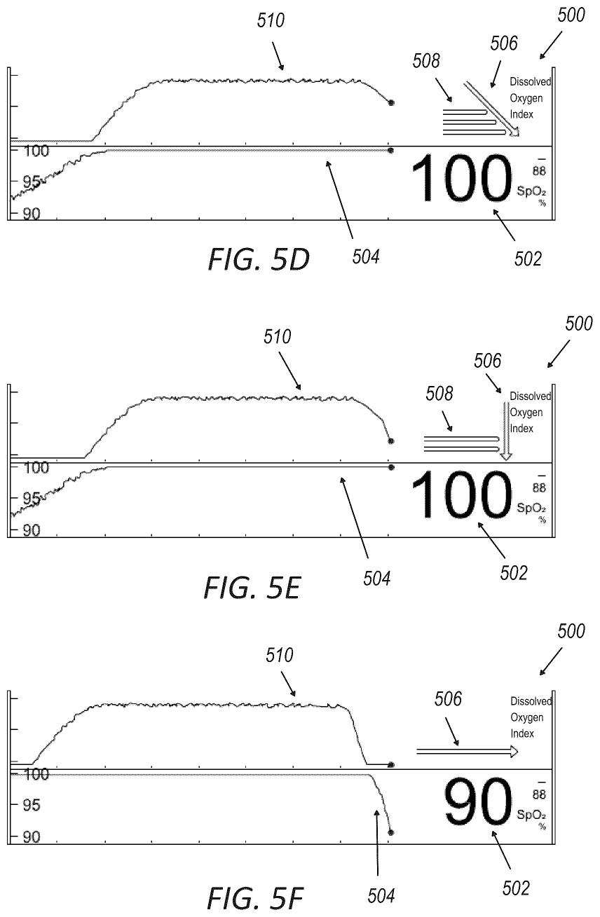

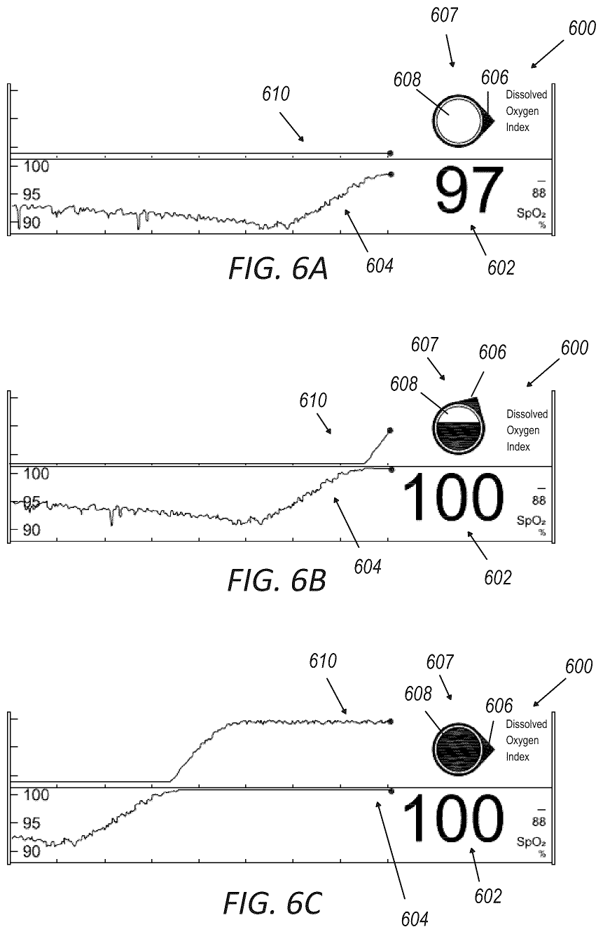

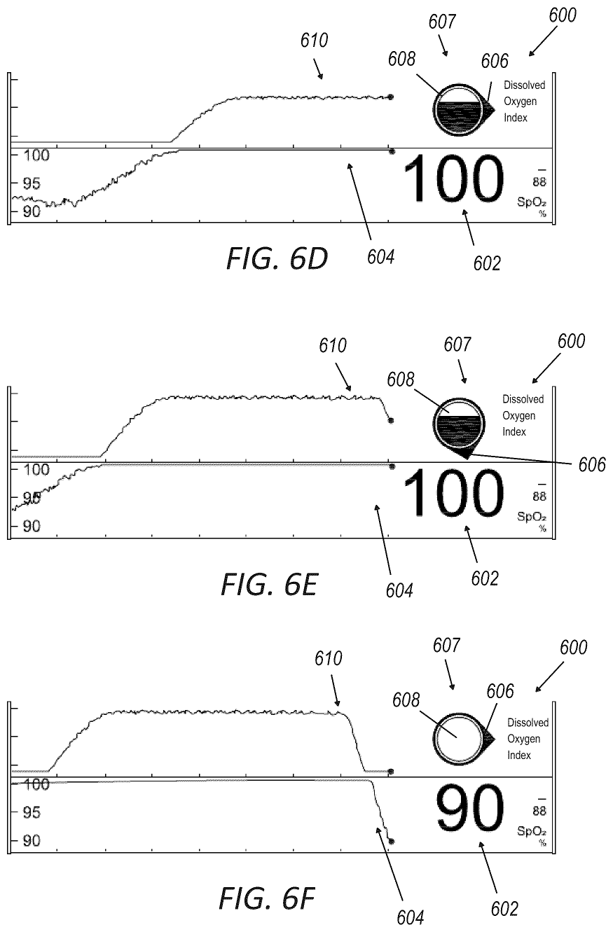

FIGS. 5A-5F and 6A-6F illustrates example displays or a portion thereof having a user-interface for displaying indications of a patient's oxygen state. The displays 500, 600 can be the display 108, 208 of FIGS. 1A and 1B, the display 304 of FIGS. 2A and 2B, or any other display for displaying physiological parameters. The indications displayed can include SpO₂, the index, and/or an increasing or decreasing trend of the index. SpO₂ can be displayed as a numerical value 502, 602 and/or a graph 504, 604 with time as the horizontal axis.

As shown in FIGS. 5A-5F, the index can be displayed as a plurality of bars 508 and/or a graph 510 with time as the x-axis. The SpO₂ graph 504 and/or the index graph 510 can be plotted along the same horizontal axis. For example, the SpO₂ graph 504 can be plotted below the horizontal axis and the index graph 510 can be plotted above the horizontal axis, as shown in the illustrated example. The graphs 504 and 510 can also be superposed on each other or the graph 504 can be above the horizontal axis and the graph 510 can be below the horizontal axis. Using the same horizontal axis can reduce a display area needed for displaying both graphs 504 and 510, and/or allow direct comparison of the SpO₂ and index graphs 504, 510.

The plurality of bars 508 can be stacked generally horizontally, vertically or at any other angle. As shown in FIGS. 5A and 5F, the display 500 does not show any bars when SpO₂ is below a threshold level. The threshold level can be about 97%, about 98%, or any other level. The threshold level can vary depending on ambient condition and/or patients. The index graph 510 can be a substantially flat baseline in FIGS. 5A and 5F.

As shown in FIGS. 5B to 5E, SpO₂ is above the threshold level and a plurality of rectangular bars 508 are displayed. The number of bars 508 can correspond to an instantaneous index level as shown by a dot in the index graph 510 and can inform the user of the patient's current oxygen reserve. The displays 500 in FIGS. 5B and 5E show two bars. The display 500 in FIG. 5D shows three bars. The display 500 in FIG. 5C shows four bars. Accordingly, the instant index level can be the highest in FIG. 5C and the lowest in FIGS. 5B and 5E. Four bars can be the maximum number of bars so that the index level is at the maximum in FIG. 5C, although other number of bars can be displayed to indicate the maximum index level. The plurality of bars 508 can have the same or different colors. The plurality of bars 508 can also optionally have the same or different shapes and/or sizes. For example, the display 500 can show a plurality of circles, triangles, diamonds, or any other shapes.

The increasing or decreasing trend of the index can be displayed as an arrow 506. The trend of the index can represent the instantaneous gradient of the graph 510. The arrow 506 can be pointing at an angle. The direction of the arrow can correspond to the increasing or decreasing trend of the patient's index. As shown in FIGS. 5A and 5F, when no index bar is shown and/or when the index graph 510 is a substantially flat baseline, the arrow 506 can be pointing generally horizontally. In FIGS. 5B and 5C, when the index is increasing as shown by the slope of the index graph 510, the arrow 506 can be pointing generally upward. In FIGS. 5D and 5E, when the index is decreasing as shown by the slope of the index graph 510, the arrow 506 can be pointing generally downward. The generally upwardly pointing arrow 506 can be on the left hand side of the plurality of bars 508. The generally downwardly pointing arrow 506 can be on the right hand side of the plurality of bars 508. Alternatively, the arrow 506 can always be displayed in the same location relative to the plurality of bars 508, such as on the left, right hand side, on the top, or below the plurality of bars 508.

A magnitude of the angle at which the arrow 506 points can correspond to a rate of increase or decrease of the patient's index. In FIGS. 5B, 5C, and 5D, the index level is increasing or decreasing at a lower rate than the increase or decrease of the index level in FIG. 5E, respectively, as shown by the slope of the corresponding index graph 510. Accordingly, the arrow 506 in FIG. 5E is steeper than the arrow 506 in FIGS. 5B, 5C, and 5D. The rate of increase or decrease of the patient's index can be useful. For example, a clinician to estimate the amount of time before the patient returns from a hypersaturated state to a baseline saturation state based on how quickly the index changes.

As further shown in FIGS. 5B to 5E, the plurality of bars 508 can have incrementally greater lengths. The varying lengths of the plurality of bars 508 can accommodate the arrow 506 at the angle. When the angle of the arrow 506 is small, the increments in the lengths of the plurality of bars 508 can be big. When the angle of the arrow 506 is larger, the increments in the lengths of the plurality of bars 508 can be smaller. The varying lengths of the plurality of bars 508 can allow the display of the plurality of bars 508 and the arrow 506 to be more compact, thereby reducing a display area required for the plurality of bars 508 and the arrow 506. The varying lengths of the plurality of bars 508 can also provide visual indication of the rate of increase or decrease of index. For example, when the plurality of bars 508 are shown as having substantially the same length, the index level can be rapidly increasing or decreasing.

Turning to FIGS. 6A to 6F, the index can be displayed as a circle 608 and/or a graph 610 with time as the horizontal axis. The SpO₂ graph 604 and the index graph 610 can be plotted along the same horizontal axis. For example, the SpO₂ graph 604 can be plotted below the horizontal axis and the index graph 610 can be plotted above the horizontal axis, as shown in the illustrated example. The graphs 604 and 610 can also be superposed on each other or the graph 604 can be above the horizontal axis and the graph 610 can be below the horizontal axis. Using the same horizontal axis can reduce a display area for displaying both graphs 604 and 610, and/or allow direct comparison of the SpO₂ and the index graphs 604, 610. The SpO₂ and index graphs 504, 604, 510, 610 can be optional, such as when the display area of a patient monitoring device is too small and/or crowded, or when a user selects a display mode in which the graphs are hidden.

The circle 608 can be empty, partially filled, or fully filled. The circle can also be a triangle, a rectangle, a diamond, or any other shape. As shown in FIGS. 6A and 6F, the circle 608 is substantially empty when SpO₂ is below a threshold level. The threshold level can be about 97%, about 98% or any other suitable level. The graph 610 can be a substantially flat baseline in FIGS. 6A and 6F.

As shown in FIGS. 6B to 6E, SpO₂ is above the threshold level and the circle 608 is at least partially filled. The amount or extent of filling of the circle 608 can correspond to an instantaneous index level as shown by a dot in the index graph 610 and can inform the user of the patient's current oxygen reserve. FIGS. 6B, 6D, and 6E show the circle 608 as partially filled. The circles 608 in FIGS. 6D and 6E are slightly more fully filled than the circle 608 in FIG. 6B. FIG. 6C shows the circle 608 as substantially fully filled. Accordingly, the instant index level can be the highest in FIG. 6C and the lowest in FIG. 6B. When the circle 608 is substantially fully filled, the patient's oxygen state may have exceeded mild hyperoxia as defined herein and may be in more severe hyperoxia.

The increasing or decreasing trend of index can be displayed as a pointer 606. The trend of index can be the instantaneous gradient of the index graph 610. The pointer 606 can be pointing at an angle. The direction of the pointer 606 can correspond to the increasing or decreasing trend of the patient's index. As shown in FIGS. 6A and 6F, when the circle 608 is substantially empty and/or when the index graph 610 is a substantially flat baseline, the pointer 606 can be pointing generally horizontally. In FIGS. 6B and 6C, when index is increasing as shown by the slope of the index graph 610, the pointer 606 can be pointing generally upward. In FIGS. 6D and 6E, when index is decreasing as shown by the slope of the graph 610, the pointer 606 can be pointing generally downward.

In FIGS. 6A to 6F, the pointer 606 can be located along the perimeter of the circle 608. A dial 607 can be formed by the circle 608 and the pointer 606. A radial or clock position of the pointer 606 can correspond to an increasing or decreasing trend of the index and/or a rate of the increase or decrease. In FIGS. 6A, 6C, 6D, and 6F, where index is leveling off as indicated by the substantially flat line of the index graph 610, the pointer 606 is at about the three o'clock. In FIG. 6B, where the index level is increasing as indicated by an upward slope of the index graph 610, the pointer 606 is at about the one o'clock. In FIG. 6E, where the index level is decreasing rapidly as indicted by the steep downward slope of the index graph 610, the pointer 606 is at between about five o'clock and about six o'clock.

The displayed indications of SpO₂, the index, and/or an increasing or decreasing trend of index on the same display can provide direct visual information of the patient's oxygen state or other physiological parameters. The visual information can be easy to understand by a user upon glancing at the display and/or can reduce the need for the user to further mentally process the oxygen state indications.

In FIGS. 5A and 6A, the patient's index is not displayed when the patient's SpO₂ is below a threshold. The threshold can be about 97%, about 98%, or any other suitable level. The threshold can be adjusted based on the patient, the medical procedure being performed, and/or ambient conditions. A user viewing the display 500, 600 in FIGS. 5A and 6A can interpret the readings to indicate that the patient is in a normoxic state. A user of the patient monitoring system, such as a clinician, can safely provide supplemental oxygen, or continue to provide supplemental oxygen, to the patient monitored by the system.

In FIGS. 5B and 6B, the patient's SpO₂ is at its maximum. The patient's index is at a moderate level and is increasing. The user viewing the display 500, 600 in FIGS. 5B and 6B can interpret the readings to indicate that patient is in mild hyperoxia as defined herein. The user may reduce, slow down, or stop supplemental oxygen delivery to the patient.

In FIG. 5C, the patient's SpO₂ is at its maximum. The patient's index is also at its maximum and is still increasing. The user viewing the display 500 in FIG. 5C can interpret the readings to mean that patient is in more severe hyperoxia. The user may need to stop oxygen delivery to the patient immediately. The user may also consider additional interventional procedures to stop or mitigate damages to the patient caused by the more severe hyperoxia.

In FIG. 6C, the patient's SpO₂ and the index are still at their maximum. However, the patient's index has stopped increasing and has leveled off. In some implementations, the leveling of index can be caused by the user cutting off supplemental oxygen delivery to the patient.

In FIG. 5D, the patient's SpO₂ is still at its maximum. However, the patient's index is no longer at its maximum and is decreasing. The patient is back in the mild hyperoxic state as defined herein. This can be cause by reduced or cutting off of supplemental oxygen delivery. The user may consider resuming supplemental oxygen delivery.

In FIG. 6D, the patient's SpO₂ is still at its maximum. The patient's index is no longer at its maximum, and is neither increasing nor decreasing. This can be an oxygen state that the user wants to maintain for the patient, such as an adult patient.

In FIGS. 5E and 6E, although the patient's SpO₂ is still at its maximum, the patient's index is no longer at its maximum and is rapidly decreasing. This can be warning of impending hypoxia. As described above, SpO₂ alone may be inadequate and/or lack sufficient sensitivity in providing warning of impending hypoxia. The user may need to resume supplemental oxygen delivery or increase the amount of oxygen delivery to the patient.

In FIGS. 5F and 6F, the patient is SpO₂ is below an optimal oxygen saturation level and the patient's index is not displayed. The patient can be in hypoxia. The user must resume supplemental oxygen delivery or increase the amount of oxygen delivery to the patient. The user may also need to consider additional interventional procedures to stop or mitigate damages to the patient due to hypoxia.

The displays as shown in FIGS. 5A-5F and 6A-6F can save time needed for and/or improve accuracy in deciding the amount of supplemental oxygen that needs to be delivered to the patient. The patient's safety during the medical procedure can be improved due to better management of supplemental oxygen delivery. Certain damages, including irreversible damages, to the patient due to hypoxia and/or hyperoxia can be reduced and/or avoided. The improved efficiency and accuracy can be critical for survival of patients, such as patients in the intensive care unit or major invasive surgeries.

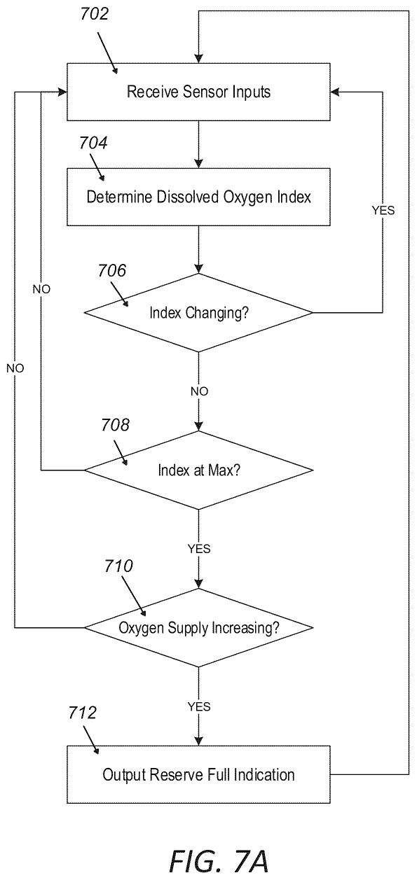

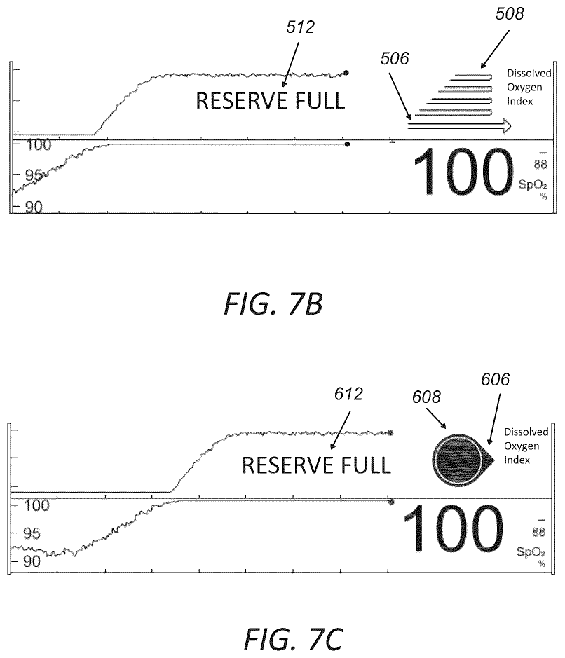

In some implementations, the processor of the patient monitoring system can monitor the plateauing of the index. The processor can output alert(s) of the plateauing of the index. As shown in FIG. 7A, at step 702 the processor can receive sensor inputs from the sensor(s) coupled to the system. At step 704, the processor can determine a plurality of physiological parameters based on the sensor input, including but not limited to the dissolved oxygen index, oxygen saturation, or otherwise. At decision step 706, the processor can determine whether the calculated dissolved oxygen index is changing. The processor can determine that the index is changing when fluctuations in the index exceeds a predetermined percentage of the previously calculated index value, such as about 1%, about 2%, about 5%, about 10%, or any ranges between those values, and that the index is not changing when the fluctuations are within the predetermined percentage. The processor can also optionally determine that the index is not changing after the index remains substantially same for a predetermined period of time, such as about 1 second, about 3 seconds, about 5 seconds, about 10 seconds, or any value between those ranges. The delay can allow the processor to ignore temporarily stationary values of the index, which may not be indicative of the physiological state of interest, such as the plateauing of the index. If the index is determined to be changing, the processor can return to step 702 to continue receiving inputs from the coupled sensors. If the index is determined to be not changing, at decision step 708, the processor can determine whether the index is at its maximum value and/or when the index is plateauing at its maximum value. If the index is not changing and not at its maximum value, the processor can return to step 702 to continue receiving inputs from the coupled sensors. If the index is plateauing at its maximum value, at step 710, the processor can determine whether oxygen supply to the patient is increasing. If the oxygen supply to the patient is not increasing (such as when oxygen supply to the patient has been stopped or decreased), the processor can return to step 702 to continue receiving inputs from the coupled sensors. If the oxygen supply to the patient is increasing (for example, if the fraction of oxygen inspired (FiO₂) is increasing, the flow rate of oxygen to the patient is increasing, or otherwise), at step 712, the processor can output an indication of the plateauing of the dissolved oxygen index.

The indication of the plateauing of the dissolved oxygen index can be presented in one or more forms, such as via a visual indicator, text message, and/or audio message. FIGS. 7B and 7C illustrate examples of display of the indication. As shown, a text message of “RESERVE FULL” 512, 612 can be displayed in the display area for the dissolved oxygen index when the index is plateauing and the oxygen supply is still increasing. FIG. 7B also illustrates a maximum number (for example, four, five, or more) of bars 508 and a generally horizontally pointing arrow 506 when the index is plateauing and the oxygen supply is still increasing. FIG. 7C also illustrates the pointer 606 pointing generally horizontally and the circle 608 being substantially full when the index is plateauing and the oxygen supply is still increasing. Additionally or alternatively to the example displays in FIGS. 7B and 7C, the processor can also output an audio message indicating that the index is plateauing when the oxygen supply is increasing.

In one example, the “RESERVE FULL” indication can be used to indicate the completion of a nitrogen washout performed on the patient. The nitrogen washout is a test for estimating a patient's functional residual capacity of the lungs. As the anatomical dead space of the lungs are filled (such as gradually filled) with oxygen after a nitrogen washout, any addition increase in the partial pressure of oxygen, for example, due to a higher FiO₂, a higher flow rate of oxygen supply to the patient, or otherwise, can be due to more oxygen getting dissolved in the blood plasma. Accordingly, when the index does not further increase despite an increase in the oxygen supply to the patient, the nitrogen washout is likely complete on the patient. The indication can be in the form of a simple message, a visual alert, an audible alert, a warning message, or any combination of the above.

The display elements disclosed herein, such as the bar(s), arrow, circle, pointer, any of their variants, and any combinations thereof, can represent any physiological parameters, of which the dissolved oxygen index is one example. The display elements disclosed herein can be used to represent a magnitude, an increasing or decreasing trend, and/or a rate of change of any physiological parameter. When the physiological parameter is zero or outside a range that can be calculated by the patient monitoring device, the display area can show a generally horizontally pointing arrow without a bar or its equivalent, or a dial having an empty circle, or its equivalent, pointing at about 3 o'clock. Displaying indications of the magnitude and/or changes of a physiological parameter together can provide more comprehensive information about a patient's physiological conditions in a limited and/or crowded display area than displaying the magnitude and the changes of the physiological parameter separately.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may comprise, additional to its essential features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A noninvasive patient monitoring system for providing an indication of a physiological parameter, the system comprising:
one or more sensors for outputting signals in response to a patient's physiological conditions;
one or more signal processors configured to receive the signals and output:
a magnitude of a physiological parameter, and
an instantaneous trend of the physiological parameter; and
a display responsive to output of the one or more signal processors to display the magnitude and instantaneous trend of the physiological parameter including in the form of a display element, the display element including a first non-waveform part and a second non-waveform part, wherein the first non-waveform part indicates the magnitude of the physiological parameter and the second non-waveform part indicates the instantaneous trend of the physiological parameter.

2. The system of claim 1, wherein the first non-waveform part comprises a plurality of objects, a number of the plurality of objects displayed corresponding to the magnitude of the physiological parameter.

3. The system of claim 2, wherein the plurality of objects each comprise a rectangle, a circle, a triangle, or a diamond.

4. The system of claim 2, wherein the second non-waveform part comprises an arrow, the arrow being adjacent to the plurality of objects.

5. The system of claim 4, wherein an angle of the arrow corresponds to a rate of change of the physiological parameter.

6. The system of claim 1, wherein the first non-waveform part comprises an object that is empty, partially filled, or fully filled, an amount of filling of the object corresponding to the magnitude of the physiological parameter.

7. The system of claim 6, wherein the object is empty when the physiological parameter is zero or indeterminate.

8. The system of claim 6, wherein the object comprises a circle, a triangle, a rectangle, or a diamond.

9. The system of claim 6, wherein the first non-waveform part comprises a circle and the second symbol part comprises a pointer, the pointer coupled and in contact with the circle to form a dial, a clock direction of the pointer corresponding to the instantaneous trend of the physiological parameter and/or a rate of change of the physiological parameter.

10. The system of claim 5, wherein the first non-waveform part comprises a plurality of stacked bars with varying lengths so as to form a slope that accommodates the angle of the arrow.

11. The system of claim 1, wherein neither the first part nor the second part includes an alphanumeric character.

12. The system of claim 1, wherein the physiological parameter is a dissolved oxygen index.

13. The system of claim 12, wherein the one or more sensors comprise an optical sensor.
